(19) United States
(12) Patent Application Publication
(10) Pub. No.: US 2020/0222507 A1
(43) Pub. Date: Jul. 16, 2020

(54) METHOD OF PREPARING LIPID NANOCARRIER

(71) Applicant: National Tsing Hua University, Hsinchu City (TW)

(72) Inventors: Hsing-Wen Sung, Hsinchu City (TW); Po-Yen Lin, Hsinchu City (TW); Kuan-Hung Chen, Hsinchu City (TW)

(73) Assignee: National Tsing Hua University, Hsinchu City (TW)

(21) Appl. No.: 16/443,895

(22) Filed: Jun. 18, 2019

(30) Foreign Application Priority Data
Jan. 16, 2019 (TW) 108101655

(51) Int. Cl. A61K 38/26; A61K 38/28; A61K 47/24; A61K 47/14; A61K 9/107

(52) U.S. Cl. CPC A61K 38/26; A61K 38/28; A61K 9/107; A61K 47/14; A61K 47/24

(57) ABSTRACT

A method of preparing a lipid nanocarrier for encapsulating a hydrophilic protein is provided. The preparing method includes the following steps. The hydrophilic protein, a lipophilic component, and a wetting agent are mixed to obtain a homogeneous solution. The homogeneous solution and a saturated salt solution comprising a surfactant are mixed to obtain the lipid nanocarrier in a single-step emulsification manner.

### CROSS-REFERENCE TO RELATED APPLICATION

This application claims the priority benefit of Taiwan application serial no. 108101655, filed on Jan. 16, 2019. The entirety of the above-mentioned patent application is hereby incorporated by reference herein and made a part of this specification.

### BACKGROUND

### Technical Field

The disclosure relates to a method of preparing a carrier, and in particular to a lipid nanocarrier.

### Description of Related Art

The medicine for the treatment of diabetes and pancreas-related diseases is mainly hydrophilic macromolecular protein medicine (such as Exenatide). This kind of medicine is difficult to be absorbed through oral administration, and the bioavailability is defective; therefore, clinically, the medicine is delivered through subcutaneous injection. However, the physical pain and the psychological fear caused by the injection usually make patients feel repulsive to the treatment.

Therefore, how to prepare a medicine carrier that is for oral administration and has high bioavailability is an issue desired to be solved quickly by researchers.

### SUMMARY

The disclosure provides a method of preparing a lipid nanocarrier, and the lipid nanocarrier has a good bioavailability.

The disclosure provides a method of preparing a lipid nanocarrier, and the lipid nanocarrier is used for encapsulating the hydrophilic protein. The method includes: mixing the hydrophilic protein, a lipophilic component, and a wetting agent to obtain a homogeneous solution; and mixing the homogeneous solution with a saturated salt solution containing a surfactant to obtain the single emulsion lipid nanocarrier.

In some embodiments of the disclosure, the abovementioned hydrophilic protein is, for example, Exenatide, a derivative of Exenatide or insulin.

In some embodiments of the disclosure, the abovementioned lipophilic components are, for example, fatty acid, phospholipid, a derivative of phospholipid, triglyceride or an ester derivative thereof.

In some embodiments of the disclosure, the abovementioned fatty acid is, for example, caproic acid, caprylic acid, capric acid, dodecanoic acid or an isomer thereof.

In some embodiments of the disclosure, the abovementioned wetting agent is, for example, Span 60, Span 80 and a derivative of Span.

In some embodiments of the disclosure, the abovementioned surfactant is, for example, Tween 60, Tween 80 or a derivative of Tween.

In some embodiments of the disclosure, the salt in the abovementioned saturated salt solution is, for example, sodium citrate or potassium citrate.

In some embodiments of the disclosure, the concentration of the sodium citrate in the saturated salt solution is 300 g/liter to 800 g/liter.

In some embodiments of the disclosure, the weight ratio of the abovementioned hydrophilic protein to the lipophilic component and the wetting agent is 1:200:50 to 1:400:50.

In some embodiments of the disclosure, the content of the surfactant in the abovementioned saturated salt solution is 10 wt % to 15 wt %.

In some embodiments of the disclosure, the weight ratio of the abovementioned homogeneous solution to the saturated salt solution is 1:4 to 1:8.

Based on the above, the method of preparing the lipid nanocarrier of the disclosure may greatly decrease the risk of loss of the hydrophilic protein encapsulated in an oil phase to a water phase through the addition of a wetting agent and a salting out effect caused by the saturated salt solution, so as to increase the loading efficiency and the bioavailability of the hydrophilic protein in the lipid nanocarrier.

In order to make the features and advantages of the disclosure mentioned above more understandable, embodiments will be described in detail below with reference to the accompanying drawings.

### BRIEF DESCRIPTION OF THE DRAWINGS

FIG. 1 is a Transmission Electron Microscopy (TEM) view of a lipid nanocarrier of Embodiment 1.

FIG. 2 shows the loading efficiency of the lipid nanocarriers of Embodiment 1, Comparative Example 1 and Comparative Example 2.

FIG. 3 shows the particle size and the zeta potential of the lipid nanocarriers of Embodiment 1 related to time.

FIG. 4 shows the residual amount of the Exenatide in the lipid nanocarrier of Embodiment 1 in the simulated intestinal fluid (SIF) related to time.

FIG. 5 shows the residual amounts of the Exenatide in free form as well as the Exenatide in the lipid nanocarrier of Embodiment 1 both degraded by the enzyme related to time.

FIG. 6 shows a change of the particle size of the lipid nanocarrier of Embodiment 1 in the simulated gastric fluid (SGF) and SIF.

FIG. 7 shows the change of the zeta potential of the lipid nanocarrier of Embodiment 1 in the SGF and SIF.

FIG. 8 to FIG. 10 shows the biodistribution in the organs in the rats which are allocated respectively in the group that takes the empty lipid nanocarrier and the free form Exenatide through oral administration, the group that takes the free form Exenatide through subcutaneous injection, and the group that takes the lipid nanocarrier loaded with the Exenatide through oral administration.

FIG. 11 is the time profile of the plasma concentration of insulin.

FIG. 12 is the time profile of the change of glucagon in the plasma.

FIG. 13 is the time profile of the change of glucose in the plasma.

### DESCRIPTION OF THE EMBODIMENTS

The disclosure provides a method of preparing a lipid nanocarrier. The lipid nanocarrier prepared by the method has a good bioavailability, and may neither be damaged by gastric acid nor be degraded by digestive enzymes, and may avoid the first pass effect of the liver. To make the present disclosure fully understandable, the preparing steps of the lipid nanocarrier will be described in details in the following content. However, the well-known composition or preparing steps are not described in the details to avoid the limitation to the disclosure. Preferable embodiments of the disclosure would be described as follows, but the disclosure is not limited hereto, and the disclosure may be broadly applied to other embodiments, and the scope of the disclosure is not limited hereto. The scope of the disclosure is defined by the claims as follows.

The lipid nanocarrier of the disclosure is used for encapsulating the hydrophilic protein, and the preparing method includes: mixing a hydrophilic protein, a lipophilic component, and a wetting agent to obtain a homogeneous solution; and mixing the homogeneous solution with the saturated salt solution containing a surfactant to obtain the single emulsion lipid nanocarrier. In some embodiments, the hydrophilic protein is, for example, Exenatide, a derivative of Exenatide or insulin. The concentration of the hydrophilic protein is, for example, 0.06 wt % to 0.13 wt %.

In some embodiments, the homogeneous solution is an oil phase solution. In some embodiments, the lipophilic component is, for example, fatty acid, phospholipid, a derivative of phospholipid, triglyceride or an ester derivative thereof. The fatty acid is, for example, caproic acid, caprylic acid, capric acid, dodecanoic acid or an isomer thereof. In some embodiments, the wetting agent is, for example, Span 60, Span 80 or a derivative of Span. In some embodiments, the hydrophilic protein is the Exenatide, the lipophilic component is capric acid, and the wetting agent is Span 80. In some embodiments, the weight ratio of the hydrophilic protein to the lipophilic component and the wetting agent is 1:400:50. In some embodiments, the mixing method is, for example, stirring the hydrophilic protein, the lipophilic component and the wetting agent under an environment of 30° C. to 40° C. for 0.15 hour to 0.5 hour. During the mixing process, the wetting agent may be helpful for combining the hydrophilic protein and the lipophilic component.

In some embodiments, the saturated salt solution is a water phase solution. In some embodiments, the surfactant is, for example, Tween 60, Tween 80 or a derivative of Tween. The content of the surfactant in the saturated salt solution is, for example, 10 wt % to 15 wt %. In some embodiments, the salt in the saturated salt solution is sodium citrate or potassium citrate. In some embodiments, the salt in the saturated salt solution is sodium citrate, and the concentration of the sodium citrate is 300 g/liter to 800 g/liter. Within the abovementioned concentration scope, the concentration of the sodium citrate is saturated.

In some embodiments, the weight ratio of the homogeneous solution to the saturated salt solution is 1:4 to 1:8. In some embodiments, the method of mixing the homogeneous solution and the saturated salt solution is to ultrasonicate the homogeneous solution and the saturated salt solution containing the surfactant under an environment of 30° C. to 40° C. for 0.15 hour to 0.5 hour. During the mixing process, the hydrophilic protein self-emulsifies to be loaded into the lipid nanocarrier. In the present embodiment, through the assistance of the wetting agent and the salting out effect caused by the saturated salt solution, the risk of loss of the hydrophilic protein encapsulated in the oil phase to the water phase may be greatly reduced, so that the loading efficiency of the hydrophilic protein in the lipid nanocarrier is increased.

Generally, after the hydrophilic protein medicine for the treatment of pancreas-related diseases (such as diabetes) is absorbed by the small intestine through oral administration, the hydrophilic protein medicine intends to enter the liver through the hepatic portal vein and then to be distributed throughout the whole body via the systemic circulation. On another front, the lipid oil droplet or the hydrophobic medicine intends to enter the pancreas through the route of lymphatic system. According to the method of preparing the lipid nanocarrier of the disclosure, the hydrophilic protein medicine is loaded into the lipid nanocarrier through the salting out effect and emulsification, and may neither be damaged by gastric acid nor be degraded by the digestive enzymes after the oral administration. The lipid nanocarrier of the disclosure may target the hydrophilic protein medicine to the pancreas through the route of lymphatic system after being absorbed by the epithelial cell and Microfold cell (M cell) of the small intestine through the transcytosis. The medicine absorbed via oral administration is targeted to the pancreas through the route of lymphatic system, and may avoid the first pass effect of the liver. Besides, the method of preparing the lipid nanocarrier of the disclosure may use the addition of the wetting agent and the salting out effect caused by the saturated salt solution simultaneously to increase the loading efficiency of the hydrophilic protein medicine, so as to greatly increase the bioavailability of the medicine.

Embodiments would be used to specifically explain the disclosure in the following content, but the Embodiments are only used for the purpose of explanation and are not used to limit the scope of the disclosure.

### EXPERIMENTAL EXAMPLE 1

The loading efficiency of Exenatide in the lipid nanocarrier.

### Embodiment 1

Firstly, 2 mg Exenatide is dissolved in a solution of 800 mg n-capric acid and 100 mg Span 80 to obtain an oil phase mixture. After the oil phase mixture is ultrasonicated in the water bath at 37° C. for 10 minutes, the saturated sodium citrate aqueous solution (300 mg/mL, 2 mL) and 600 mg Tween 80 are added, and an ultrasonic probe is used to oscillate with 70% amplitude for 1 minute (VCX 750 sonicator, Sonics & Materials Inc., USA), and finally vortexed by a vortex mixer for 1 minute to form the lipid nanocarrier loaded with the Exenatide of Embodiment 1.

### COMPARATIVE EXAMPLE 1

Firstly, 2 mg Exenatide is dissolved in the 800 mg n-capric acid to obtain an oil phase mixture. After the oil phase mixture is ultrasonicated in the water bath at 37° C. for 10 minutes, reverse osmosis pure water (2 mL) and 600 mg Tween 80 are added, and an ultrasonic probe is used to oscillate with 70% amplitude for 1 minute (VCX 750 sonicator, Sonics & Materials Inc., USA), and finally vortexed by a vortex mixer for 1 minute to form the lipid nanocarrier loaded with the Exenatide of Comparative Example 1.

### COMPARATIVE EXAMPLE 2

Firstly, 2 mg Exenatide is dissolved in a solution of 800 mg n-capric acid and 100 mg Span 80 to obtain an oil phase mixture. After the oil phase mixture is ultrasonicated in the water bath at 37° C. for 10 minutes, reverse osmosis pure water (2 mL) and 600 mg Tween 80 are added, and an ultrasonic probe is used to oscillate with 70% amplitude for 1 minute (VCX 750 sonicator, Sonics & Materials Inc., USA), and finally vortexed by a vortex mixer for 1 minute to form the lipid nanocarrier loaded with the Exenatide of Comparative Example 2.

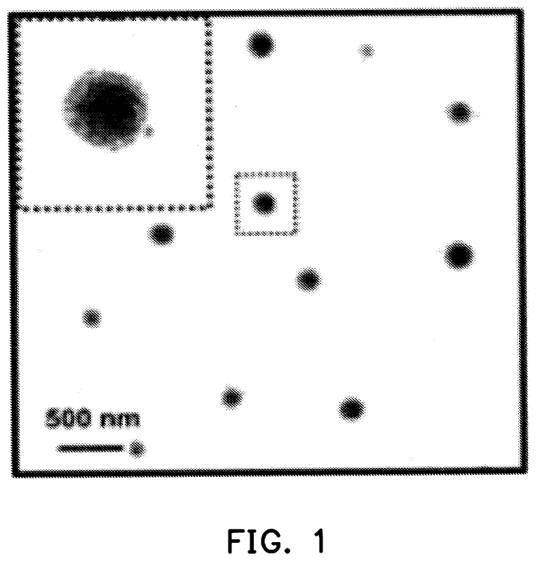

FIG. 1 is a Transmission Electron Microscopy (TEM) view of a lipid nanocarrier of Embodiment 1.

In the present Experimental Example, the medicine loading efficiency may be obtained through the following method. Firstly, the lipid nanocarriers of Embodiment 1, Comparative Example 1 and Comparative Example 2 are respectively dissolved in 2% trifluoroacetic acid (TFA), and the analysis is performed through the reverse-phase high-performance liquid chromatography (HPLC) to obtain the medicine loading efficiency. The calculation formula is as follows:

Loading efficiency (%)=The weight of the Exenatide in the lipid nanocarrier/the total weight of the added Exenatide

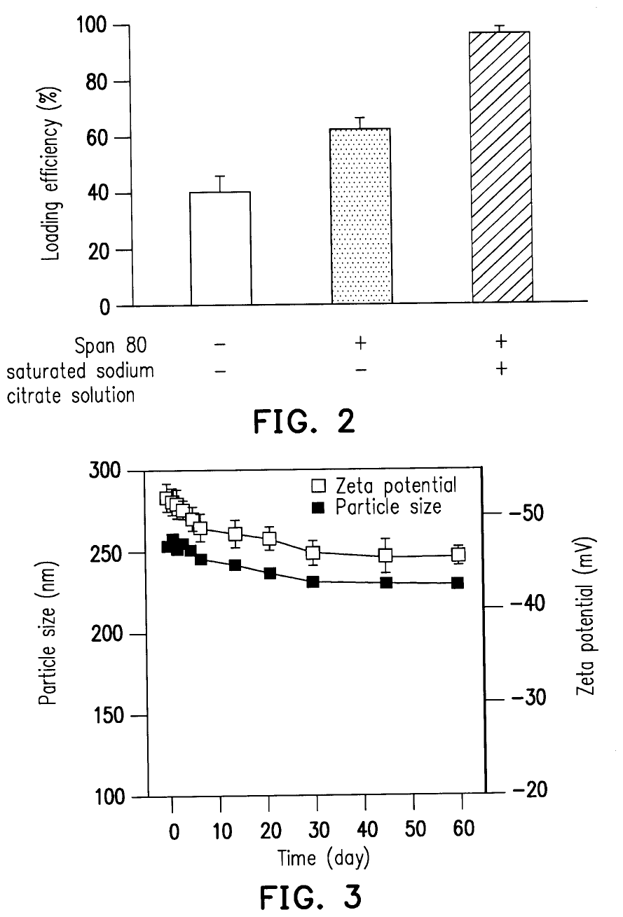

FIG. 2 shows the loading efficiency of the lipid nanocarriers of Embodiment 1, Comparative Example 1 and Comparative Example 2.

The loading efficiency of the medicine (Exenatide) of Embodiment 1 is about 97.8%. From FIG. 2, it can be known that the medicine loading efficiency of Embodiment 1 is obviously higher than the medicine loading efficiencies of Comparative Example 1 and Comparative Example 2.

In the present Experimental Example, a zetasizer (Nano-ZS) is used to further measure the particle size and Polydispersity index (PdI) of the lipid nanocarrier of Embodiment 1, and the obtained result is described in Table 1. According to the result of Table 1, it can be known that the particle size of the lipid nanocarrier of Embodiment 1 is about 250.8 nm, and the PdI is about 0.32, which shows that the distribution of the particle size of the lipid nanocarrier of the disclosure is uniform.

TABLE 1

| | Particle | Polydispersity | Loading |
|---|---|---|---|
| | Size | index | efficiency |
| | (nm) | (PdI) | (%) |
| Embodiment 1 | 250.8 ± 9.9 | 0.32 ± 0.02 | 97.8 ± 1.6 |

### EXPERIMENTAL EXAMPLE 2

[The Test of Stability of the Lipid Nanocarrier]

In the present Experimental Example, the Nano-ZS measures the Zeta potential and the particle size of the lipid nanocarrier of Embodiment 1 in different timepoints to confirm the stability of the lipid nanocarrier of the disclosure.

FIG. 3 shows the particle size and the zeta potential of the lipid nanocarriers of Embodiment 1 related to time. From the result of FIG. 3, it can be known that within the preservation time of 60 days, the particle size of the lipid nanocarrier of Embodiment 1 is between 200 nm and 300 nm, and the Zeta potential is between -40 mV and -60 mV.

### EXPERIMENTAL EXAMPLE 3

[The Protective Effect of the Lipid Nanocarrier to the Medicine]

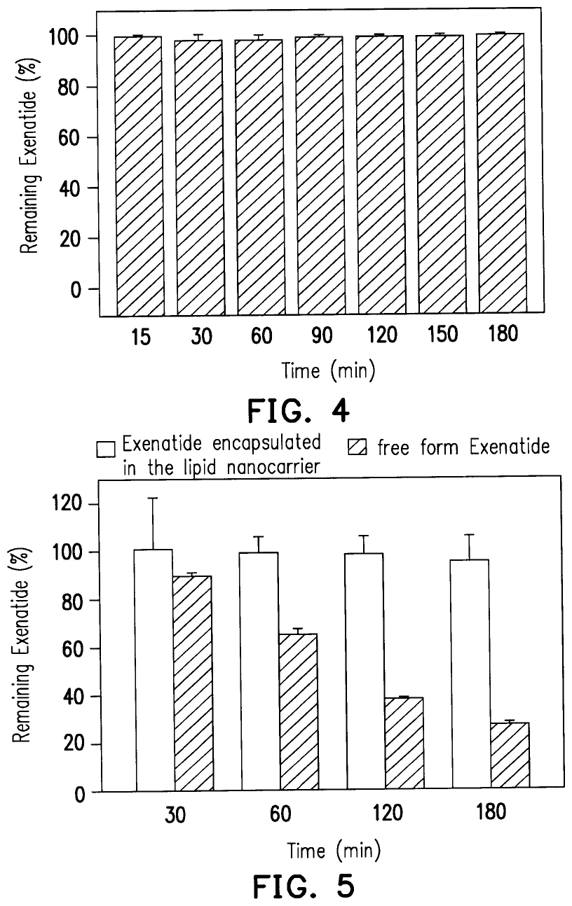

FIG. 4 shows the residual amount of the Exenatide in the lipid nanocarrier of Embodiment 1 in the simulated intestinal fluid (SIF) related to time. In the present Experimental Example, the SIF is a neutral aqueous solution (pH 7.0) containing bile extract (5 mg/mL) and lipase (1.6 mg/mL). The lipid nanocarrier of Embodiment 1 is added into the SIF and is shaken under an environment of 37° C. to measure the medicine leakage at specific timepoints (15 minutes, 30 minutes, 60 minutes, 90 minutes, 120 minutes, 150 minutes, and 180 minutes). From the result of FIG. 4, it can be known that, the lipid nanocarrier of the disclosure does not leak the medicine in the SIF, and therefore may effectively protect the medicine.

FIG. 5 shows the residual amounts of the Exenatide in free form as well as the Exenatide in the lipid nanocarrier of Embodiment 1 both degraded by the enzyme related to time. In the present Experimental Example, the Exenatide in free form and the lipid nanocarrier loaded with the Exenatide (which is the lipid nanocarrier of Embodiment 1) are respectively added into a Tris buffer solution containing 0.1 mM EDTA (ethylenediaminetetraacetic acid) and 100 μL of the abovementioned solution are added into 100 μL of a solution containing 0.5 mg trypsin and are shaken under an environment of 37° C. 200 μL of 2% trifluoroacetic acid (TFA) is added at different timepoints (30 minutes, 60 minutes, 120 minutes and 180 minutes) to end the reaction of the enzymetic degradation. Finally, HPLC is used to measure the concentration of the remaining Exenatide. From the result of FIG. 5, it can be known that the residual amount of the Exenatide in free form decreases along with the increase of the reaction time of the enzyme. However, the amount of the Exenatide in the lipid nanocarrier of Embodiment 1 still remains at almost 100% as the reaction time of enzyme increases. The result shows that the lipid nanocarrier of the disclosure may protect the medicine from being degraded by the digestive enzymes.

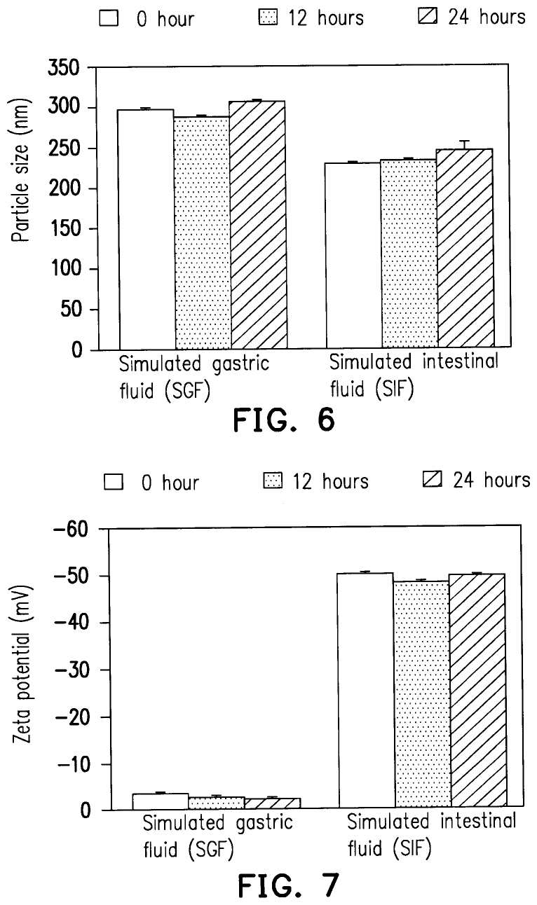

FIG. 6 shows a change of the particle size of the lipid nanocarrier of Embodiment 1 in the simulated gastric fluid (SGF) and SIF. FIG. 7 shows the change of the zeta potential of the lipid nanocarrier of Embodiment 1 in the SGF and SIF. In the present Experimental Example, the SGF is made by adding 0.5 mg/mL pepsin into a HCl aqueous solution of pH 2.0; the SIF is a neutral aqueous solution (pH 7.0) containing bile extract (5 mg/mL) and lipase (1.6 mg/mL). The lipid nanocarrier of Embodiment 1 is added into SIF and SGF respectively and is shaken under an environment of 37° C., and the particle size as well as the zeta potential are measured at specific timepoints (0 hour, 12 hours and 24 hours). From the result of FIG. 6 and FIG. 7, it can be known that the particle size of the lipid nanocarrier of Embodiment 1 in SGF and SIF does not change much. Likewise, the zeta potential of the lipid nanocarrier of Embodiment 1 in SGF and SIF does not change much. In other words, the lipid nanocarrier of the disclosure remains a stable structure in SGF and SIF.

### EXPERIMENTAL EXAMPLE 4

[The Test of Bioavailability Rate]

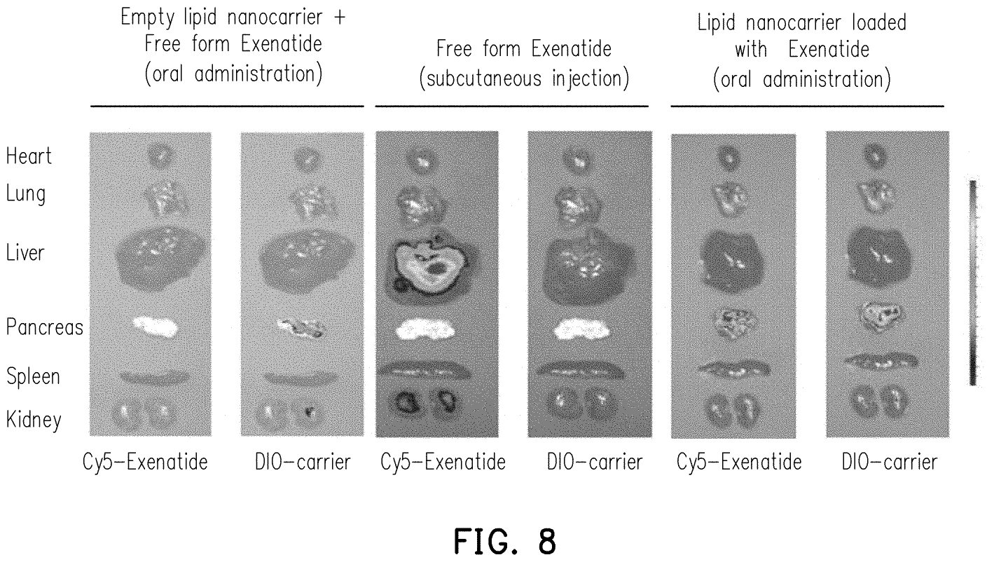

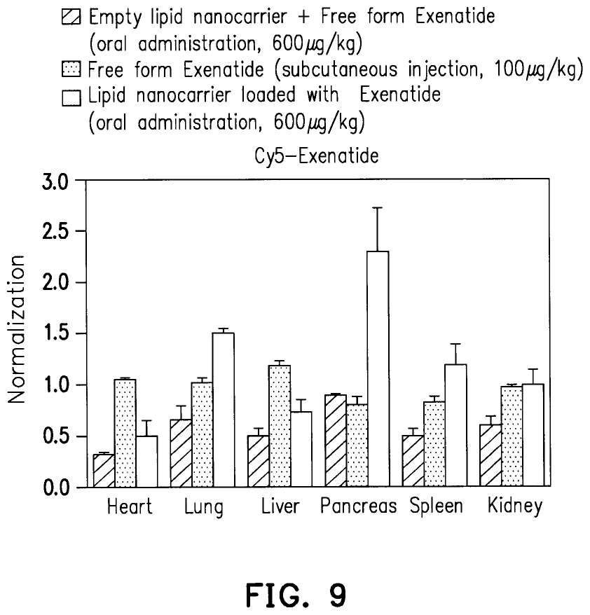

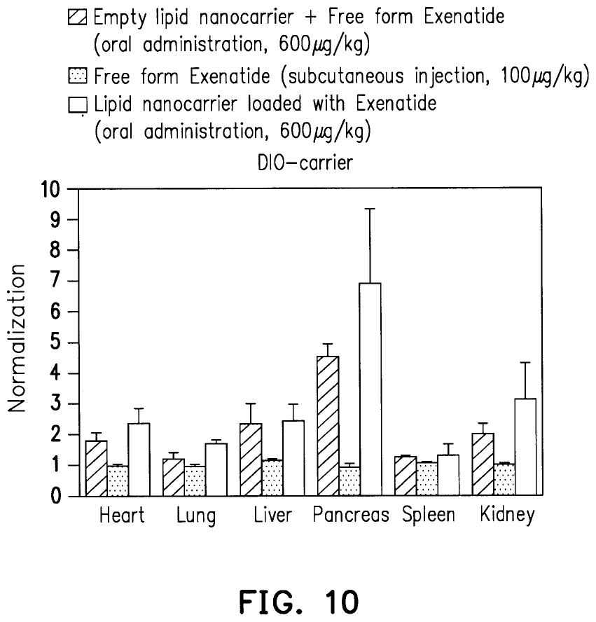

FIG. 8 to FIG. 10 shows the biodistribution in the organs in the rats which are allocated respectively in the group that takes the empty lipid nanocarrier and the free form Exenatide through oral administration, the group that takes the free form Exenatide through subcutaneous injection, and the group that takes the lipid nanocarrier loaded with the Exenatide through oral administration. In the present Experimental Example, the experimental animals are divided into the following three groups. The first group takes the empty nanocarrier and the free form Exenatide (600 μg/kg) through oral administration; the second group takes the free form Exenatide (100 μg/kg) through subcutaneous injection; the third group takes the lipid nanocarrier loaded with Exenatide (600 μg/kg) of Embodiment 1 through oral administration. Besides, in the present experiment, in order to effectively observe the biodistribution in the organs in the rats, a fluorescence-labeled Cy5-Exenatide is synthesized. Also, during the process of preparing the lipid nanocarrier, the fluorescent molecule DiO (1.0%, w/w) is added into the n-capric acid to prepare a fluorescent DiO-lipid nanocarrier, and the DiO-lipid nanocarrier may be used to load the Cy5-Exenatide. IVIS Imaging System is used to perform the observation during the experiment. In the experiments of the first and the third groups, the rats were sacrificed by carbon dioxide after 5 hours following the oral administration, and the major organs were extracted to be observed through the IVIS system. In the experiment of the second group, the free form Cy5-Exenatide is subcutaneously injected into the rat. The rat is sacrificed by carbon dioxide after one hour, and the major organs are extracted to be observed through the IVIS system. From the result of FIG. 8 to FIG. 10, it can be known that, with the assistance of the lipid nanocarrier of the disclosure, the Exenatide may obviously accumulate in the pancreas of the rat. However, under the situation that the free form Exenatide is injected through subcutaneous injection, the Exenatide may not be able to accumulate in the pancreas and may only accumulate in the liver and kidneys. In addition, under the situation that the empty lipid nanocarrier and the free form Exenatide are taken through oral administration, the Exenatide is completely not able to accumulate in the pancreas and may accumulate in the kidneys.

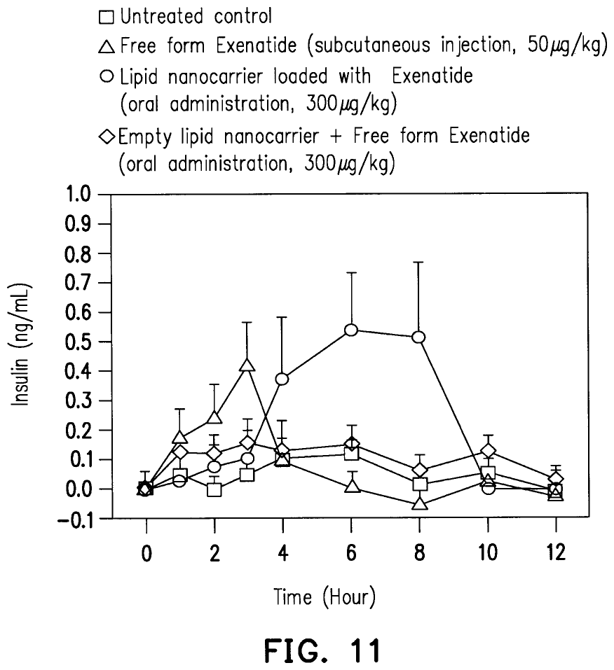

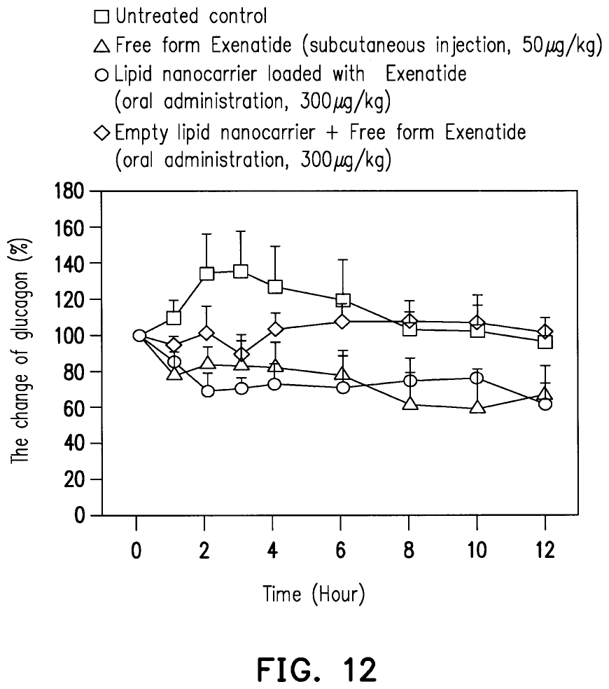

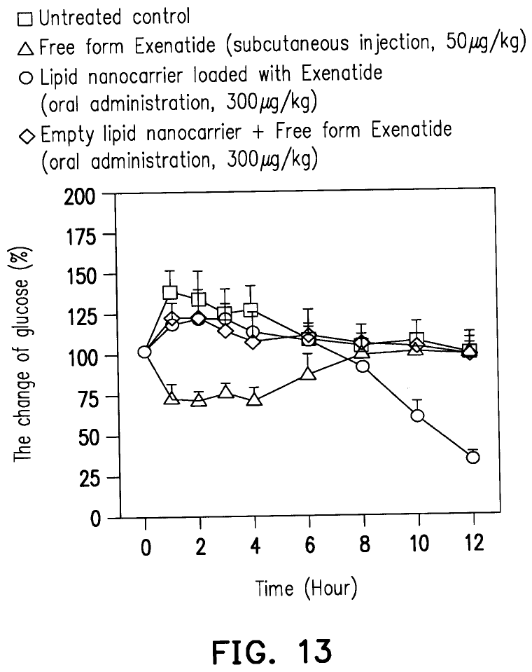

FIG. 11 is the time profile of the plasma concentration of insulin. FIG. 12 is the time profile of the change of glucagon in the plasma. FIG. 13 is the time profile of the change of glucose in the plasma. In the present Experimental Example, the rats with diabetes are used as experimental materials and the experimental animals are divided into the following four groups. The first group is a control group, which means the rats that are not treated; the second group takes the free form Exenatide (50 μg/kg) through subcutaneous injection; the third group takes the free form Exenatide and the empty lipid nanocarrier (300 μg/kg) through oral administration; the fourth group takes the lipid nanocarrier loaded with the Exenatide of Embodiment 1 (300 μg/kg) through oral administration. In the present Experimental Example, blood samples were collected from tail veins of the rats in each group. After the centrifugation, the serum samples were extracted and preserved in a refrigerator of −80° C. The changes of fasting blood glucose level of the rats were measured by the blood glucose monitor (LifeScan Inc. USA). The concentration of the insulin and glucagon in the serum were measured by Enzyme-Linked Immunosorbent Assay (ELISA).

From the results of FIG. 11 to FIG. 13, it can be known the lipid nanocarrier loaded with the Exenatide of the disclosure may be successfully delivered into the rats with diabetes through oral administration, so as to promote the secretion of insulin and suppress the secretion of glucagon, so as to further control the hyperglycemia of the rats with diabetes (which means decreasing the blood glucose of the rats with diabetes).

Although the disclosure has been disclosed in the above embodiments, the embodiments are not intended to limit the disclosure, and those skilled in the art may make some modifications and refinements without departing from the spirit and scope of the disclosure. Therefore, the scope of the disclosure is defined by the claims attached below.

What is claimed is:

1. A method of preparing a lipid nanocarrier used for encapsulating a hydrophilic protein, comprising: mixing the hydrophilic protein, a lipophilic component and a wetting agent to obtain a homogeneous solution; and mixing the homogeneous solution with a saturated salt solution comprising a surfactant to obtain the single emulsion lipid nanocarrier.

2. The method of preparing the lipid nanocarrier according to claim 1, wherein the hydrophilic protein comprises Exenatide, a derivative of Exenatide or insulin.

3. The method of preparing the lipid nanocarrier according to claim 1, wherein the lipophilic component comprises fatty acid, phospholipid, a derivative of phospholipid, triglyceride or an ester derivative thereof.

4. The method of preparing the lipid nanocarrier according to claim 3, wherein the fatty acid comprises caproic acid, caprylic acid, capric acid, dodecanoic acid or an isomer thereof.

5. The method of preparing the lipid nanocarrier according to claim 1, wherein the wetting agent comprises Span 60, Span 80 or a derivate of Span.

6. The method of preparing the lipid nanocarrier according to claim 1, wherein the surfactant comprises Tween 60, Tween 80 or a derivative of Tween.

7. The method of preparing the lipid nanocarrier according to claim 1, wherein a salt in the saturated salt solution comprises sodium citrate or potassium citrate.

8. The method of preparing the lipid nanocarrier according to claim 7, wherein a concentration of the sodium citrate in the saturated salt solution is 300 g/liter to 800 g/liter.

9. The method of preparing the lipid nanocarrier according to claim 1, wherein a weight ratio of the hydrophilic protein to the lipophilic component and the wetting agent is 1:200:50 to 1:400:50.

10. The method of preparing the lipid nanocarrier according to claim 1, wherein a content of the surfactant in the saturated salt solution is 10 wt % to 15 wt %.

11. The method of preparing the lipid nanocarrier according to claim 1, wherein a weight ratio of the homogeneous solution to the saturated salt solution is 1:4 to 1:8.
